(19) United States
(12) Patent Application Publication
(10) Pub. No.: US 2026/0325078 A1
(43) Pub. Date: Oct. 22, 2026

(54) REAL-TIME IMAGING FEEDBACK SYSTEM

(71) Applicant: Siemens Healthineers AG, Forchheim (DE)

(72) Inventors: Halid Yerebakan, Carmel, IN (US); Fabian Wagner, Erlangen (DE); Kritika Iyer, Conshohocken, PA (US)

(21) Appl. No.: 19/181,404

(22) Filed: Apr. 17, 2025

(51) Int. Cl. A61B 5/00; A61B 5/055; G06T 7/00; G06T 7/73

(52) U.S. Cl. CPC A61B 5/706; A61B 5/055; A61B 5/7405; G06T 7/0012; G06T 7/73; G06T 2207/10088; G06T 2207/20084; G06T 2207/30004; G06T 2207/30168

(57) ABSTRACT

A framework for providing real-time imaging feedback. Volumetric image of a patient is received. The framework determines an image quality measure of the volumetric image from a medical imaging device and activates a feedback device in response to the image quality measure.

### TECHNICAL FIELD

The present framework relates to automated processing of volumetric medical images.

### BACKGROUND

Positioning of the patient impacts the quality of magnetic resonance (MR) image acquisition, especially in extremities. Proper positioning not only facilitates capturing clear images and minimized motion artifacts but also contributes to patient comfort during the procedure. There are protocols to plan a full power scan with automatic alignment to detect the best orientation before doing actual scanning. These may be performed with a scout scan and automated landmark analysis.

A conventional way of matching the coordinates (or locations) between medical images is by applying registration algorithms. Rigid registration algorithms calculate rotation and translation matrices to the images to allow mapping coordinates between coordinate systems with matrix multiplications. On the other hand, non-rigid transformations allow more flexible non-linear relationships for those mappings. In both cases, the image information should have enough overlap for accurate registration. A disadvantage of these conventional registration methods is prohibitive runtime due to a large amount of computation. These registration algorithms map all the points in the corresponding images, even only a few points may be needed to be mapped in certain applications.

Landmarking methods may provide semantic location information in a single source image. One exemplary landmarking method uses several cascaded Adaboost models which can classify voxels in an image as containing a given landmark in a coarse-to-fine manner. After detecting several landmarks using Adaboost, a voting model based on the spatial correlation of landmark groups is used to remove outliers and interpolate missing landmarks. Optionally, the Adaboost and voting steps are repeated after aligning the image to a canonical space. Training the Adaboost and voting models requires at least 50-100 annotations per landmark. Once the classifier is trained, a landmark is found by scanning the entire image which takes about half a second per landmark. As a result, for 10-20 landmarks the time would be slow to give immediate feedback to adjust orientation. Instead, the method is used as post processing step after scout scan to adjust for full scan once.

These landmarking methods are relatively fast compared to registration methods. However, increasing the number of landmarks increases the required amount of work for training, while decreasing the runtime speed. In other words, there is an inherent tradeoff between the granularity of landmarks and speed. Current landmarking methods may fail in some cases where the positioning of the body part deviates from expected variations on landmark detection. Thus, it would be additional effort for technicians to either re-orient the patient or manually adjust scanning parameters.

### SUMMARY

Described herein is a framework for providing real-time imaging feedback. Volumetric image of a patient is received. The framework determines an image quality measure of the volumetric image, and activates a feedback device in response to the image quality measure.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 is a block diagram illustrating an exemplary computer system;

FIG. 2 shows an exemplary imaging feedback method;

FIG. 3 shows an exemplary method of estimating a patient's position;

FIG. 4 illustrates the exemplary iterative process of estimating the position of a landmark on a patient's foot; and

FIG. 5 shows a cross-sectional view of an exemplary medical imaging device.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance. Independent of the grammatical term usage, individuals with male, female or other gender identities are included within the term.”

Unless stated otherwise as apparent from the following discussion, it will be appreciated that terms such as “segmenting,” “generating,” “registering,” “determining,” “aligning,” “positioning,” “processing,” “computing,” “selecting,” “estimating,” “detecting,” “tracking” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present framework are not described with reference to any particular programming language. It will be appreciated that a variety of programming languages may be used.

One aspect of the present framework provides immediate feedback on image quality of a volumetric image. An image quality measure may be based on, for example, the position of the patient. The position of the patient may be estimated using a supervised neural network regression model. The supervised neural network regression model uses landmark locations as origins and regresses coordinates accordingly. Additionally, in order to speed up the regression network, sparse sampling methodology may be used. The present supervised regression model advantageously enables fast landmark estimations, allowing feedback of the image quality measure to be provided continuously and substantially in real-time. A feedback device may be used to provide the feedback to the user or technician. Once the feedback device indicates that the image quality measure is acceptable, a full power scan of the patient may be initiated. These and other features and advantages will be described in more detail herein.

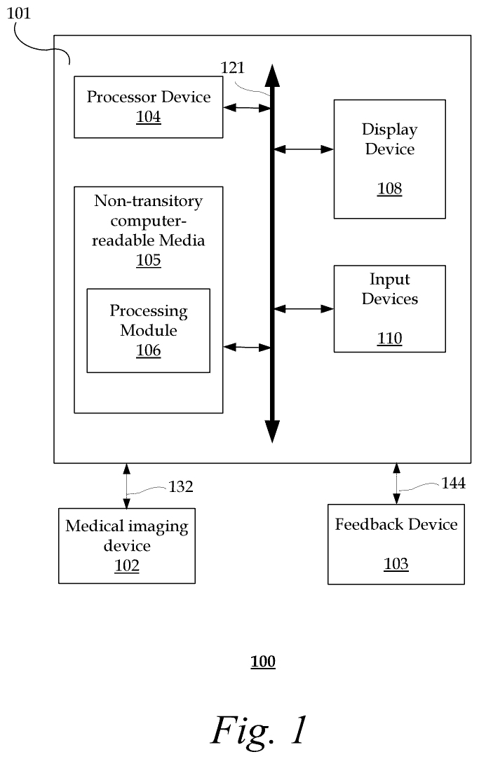

FIG. 1 is a block diagram illustrating an exemplary computer system 101 for implementing the framework as described herein. In some implementations, computer system 101 operates as a standalone device. In other implementations, computer system 101 is connected to other machines, such as medical imaging device 102 and feedback device 103. In a networked deployment, computer system 101 may operate as a peer machine in a peer-to-peer (or distributed) network environment. Any number of computer systems 101 may be provided (e.g., one, two, three or more) to serve one or more medical imaging devices 102.

Computer system 101 may include a processor device or central processing unit (CPU) 104 coupled to one or more non-transitory computer-readable media 105 (e.g., computer storage or memory device), display device 108 and input devices 110 (e.g., mouse, touchpad or keyboard) via an input-output interface 121. Computer system 101 may further include support circuits such as a cache, a power supply or battery, clock circuits and a communications bus (not shown). Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in one or more non-transitory computer-readable media 105. In particular, the present techniques may be implemented by a processing module 106. Non-transitory computer-readable media 105 may include random access memory (RAM), read-only memory (ROM), magnetic floppy disk, flash memory, and other types of memories, or a combination thereof. The computer-readable program code is executed by processor device 104 to process data 132 acquired by, for example, medical imaging device 102. The computer-readable program code is not intended to be limited to any particular programming language and implementation thereof. It will be appreciated that a variety of programming languages and coding thereof may be used to implement the teachings of the disclosure contained herein. The same or different computer-readable media 105 may be used for storing a database.

Medical imaging device 102 acquires medical image data 132. Such medical image data 132 may be processed by processing module 106. Medical imaging device 102 may be a radiology scanner and/or appropriate peripherals (e.g., keyboard, display device) for acquiring, collecting and/or storing such medical image data 132. Medical imaging device 102 may acquire medical image data 132 from a subject or patient by using techniques such as high-resolution computed tomography (HRCT), magnetic resonance (MR) imaging, computed tomography (CT), helical CT, X-ray, angiography, positron emission tomography (PET), fluoroscopy, ultrasound, single photon emission computed tomography (SPECT), or a combination thereof. Other types of imaging modalities are also useful. Medical imaging device 102 may be controlled using a medical imaging software.

Feedback device 103 provides user feedback in response to one or more image quality measures 144 determined by processing module 106. In some implementations, feedback device 103 is positioned at the medical imaging device 102 to provide feedback to the operator in response to an image quality measure. Feedback device 103 may provide, for example, visible feedback, audible feedback, tactile (or haptic) feedback, or combination thereof. A visible feedback device includes, for example, a colored light (e.g., red and green light) or a display device. An audible feedback device includes, for example, an alarm that beeps when the image quality measure meets a pre-determined criterion. A tactile feedback device may include, for example, a vibration motor that vibrates when the image quality measure meets a pre-determined criterion. Other types of feedback devices are also useful.

It is to be further understood that, because some of the constituent system components and method steps depicted in the accompanying figures can be implemented in software, the actual connections between the system components (or the process steps) may differ depending upon the manner in which the present framework is programmed. Given the teachings provided herein, one of ordinary skill in the related art will be able to contemplate these and similar implementations or configurations of the present framework.

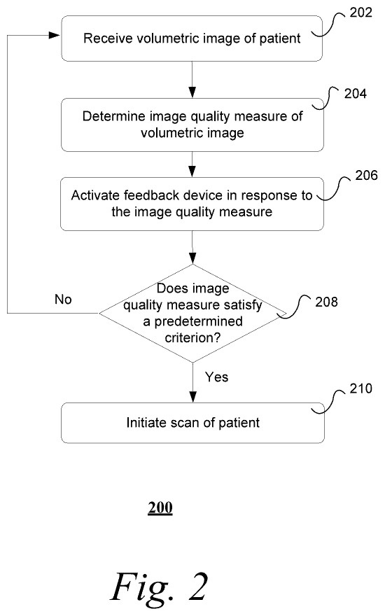

FIG. 2 shows an exemplary imaging feedback method 200. It should be understood that the steps of the method 200 may be performed in the order shown or a different order. Additional, different, or fewer steps may also be provided. Further, the method 200 may be implemented with the system 100 of FIG. 1, a different system, or a combination thereof.

At 202, processing module 106 receives a volumetric image of a patient. The volumetric image may be acquired by and/or received from, for example, medical imaging device 102. The volumetric medical image may be three-dimensional (3D) and/or related to a volume. The volumetric medical image may be made up of a number of slices, i.e., 2D (two-dimensional) medical images. The 2D medical images may be assembled to form the volumetric medical image. An anatomical part (e.g., organ, bone, joint) of the patient may be imaged. The anatomical part is to be understood as a collection of tissues joined in a structural unit to serve a common function.

The patient may be positioned on a patient support apparatus during scanning by the medical imaging device 102. In some implementations, the patient support apparatus is a table providing a single fixed planar surface for the patient to lie on. The emission camera is provided within the inner walls of a chamber formed within a tunnel shaped structure. The table is mobile for being received within the chamber. By rotating the emission camera relative to the patient, the emission camera images the patient at a variety of angles for acquiring image data in three dimensions. Other types of patient support apparatus and medical imaging devices, such as a chair-type support apparatus, may also be used.

At 204, processing module 106 determines an image quality measure of the volumetric image. Image quality measure may be a quantitative score of image quality that is determined and analyzed for how it impacts diagnostic accuracy and performance. In some implementations, the image quality measure is based on the distance of the estimated position of the patient from a desired (or expected) position. Other types of image quality measures, such as presence of motion artifacts in the volumetric image, may also be used. Different types of image quality measures may be combined to generate an overall image quality measure.

The desired position may be defined according to the type of region of interest to be imaged and/or the applicable imaging protocol to obtain the best quality image. For example, in the case where the region of interest is an Archilles tendon, the MR imaging protocol includes acquiring images in at least two orthogonal planes. The desired positions may then be defined on, for example, the sagittal and axial planes of the foot for the first and second scans. Other positions or orientations are also possible. Additionally, or alternatively, the desired position is defined at the center of the scan region.

In some implementations, the position of the patient is estimated by using a trained neural network regression model to find one or more anatomical landmarks in the volumetric image. In order to speed up the regression network, sparse sampling methodology may be used. The sparse sampling methodology is a data selection method that eliminates most of the data by selecting intensity values from sparse grids with different resolutions. The neural network regression model may be supervised to improve precision of estimated landmark positions, while maintaining the fast speed of estimation to enable continuous monitoring of patient positions in volumetric images acquired over time and provide immediate feedback beyond using a single scout image. The trained regression model uses landmark locations as origins and regresses coordinates accordingly.

In order to find a landmark, the regression model agent starts from an initial point and travels towards the landmark (i.e., the origin) during the estimation process. The initial point may be located at, for example, a center of the image or a random position. Multiple regression model agents starting at different initial points may be used to provide redundancy. Each regression model agent updates its own current position with the respective estimated landmark position after each iteration. Estimations of the landmark position may be performed for a predetermined number of iterations or until a predetermined termination criteria is met.

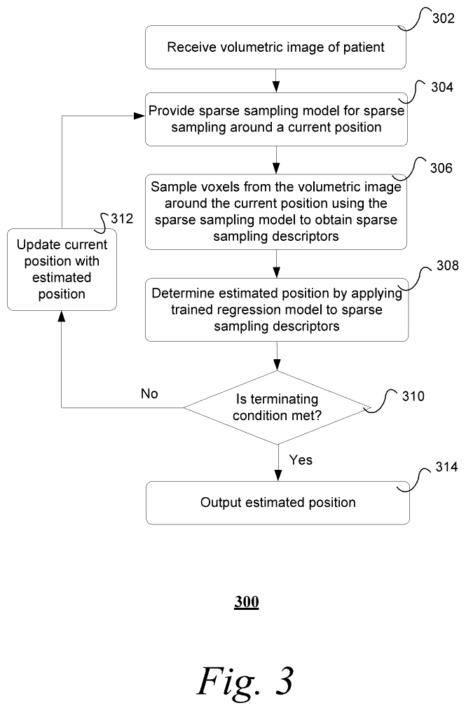

FIG. 3 shows an exemplary method 300 of estimating a patient's position. It should be understood that the steps of the method 300 may be performed in the order shown or a different order. Additional, different, or fewer steps may also be provided. Further, the method 300 may be implemented with the system 100 of FIG. 1, a different system, or a combination thereof.

At 302, processing module 106 receives the volumetric image of the patient. The volumetric image may be acquired by and/or received from, for example, medical imaging device 102, as previously described. At least one anatomical landmark indicative of the patient's position may be visible in the volumetric image. In some implementations, the anatomical landmark is a bone landmark. Other types of landmarks are also possible.

At 304, processing module 106 provides a sparse sampling model for sparse sampling around a current position in the volumetric image. “Sparse sampling” is to be understood as, with regard to the total number of voxels making up the volumetric medical image, only few voxels being used in sampling. In particular, “sparse” is to say that less than 50% or less than 20% or even less than 10% of the total number of voxels of the volumetric medical image are sampled. Sparse sampling has the effect that the amount of data which needs to be processed by the trained regression model is reduced, thus reducing computation time and computation resources.

The sparse sampling model defines a number N of sampling points around a current position. The sampling points are distributed in the volumetric image and defining locations and distances of the distributed sampling points. In some implementations, the sparse sampling model defines a plurality of grids, in particular 3D grids, of different grid spacings, the different grid spacings determining different distances of the distributed sampling points in the volumetric medical image. The respective grid spacing is defined as the distance between two adjacent nodes of the respective grid, wherein the respective node is defined as the intersection of three grid lines and forms a sampling point.

The current position may be initially set to, for example, a center of the volumetric image, and updated at step 312 before the next iteration of step 304. Other initial points, such as one that is selected by a user or a random position, may also be used. Multiple initial positions may also be used to provide redundancy. The current position may be described using three-dimensional coordinates in x, y, z and may correspond to a specific voxel in the volumetric image. In some implementations, the sparse sampling model is provided such that the voxels are sampled with a sampling rate per unit length, area or volume which decreases with a distance of the respective voxel from the current position. The sampling rate may decrease at a nonlinear rate, such as an exponential, logarithmic or power function rate. There may also be multiple current positions to provide redundancy using multiple sparse sampling models for multiple regression model agents.

At 306, processing module 106 samples voxels from the volumetric image around the current position using the sparse sampling model to obtain N sparse sampling descriptors. Sparse sampling descriptors (also referred to as point matching descriptors) provide additional speed gains due to a lower dimension. The respective sparse sampling descriptor may be formed as a vector of values, in particular, of intensities, of the sampled voxels associated with a certain sampling point of the distributed sampling points.

At 308, processing module 106 determines the estimated position of the patient by applying a trained regression model to the sparse sampling descriptors. In some implementations, the regression model is a residual neural network that is trained to embody a normalized coordinate estimator for matching between locations in the volumetric medical image and locations in a certain reference coordinate system.

To train the regression model, sparse sampling descriptors are populated from random locations and locations nearby anatomical landmarks in training volumetric data. The anatomical landmarks may be defined according to the expected orientation of the patient. Displacements to the anatomical landmarks are calculated as ground truth. Accordingly, a residual network may be trained using pairs of input descriptors and output displacements. The output displacements are used to supervise the training of the residual network. During runtime, processing module 106 applies the trained regression model to the sparse sampling descriptors to generate the normalized position of the patient. In some implementations, the output of the trained regression model are the normalized coordinates of the at least one anatomical landmark indicative of the estimated position of the patient.

At 310, processing module 106 determines whether the terminating condition is met. In some implementations, the terminating condition is whether a predetermined number of iterations have been performed. Since initial positions may be far from the target landmark, steps 304, 306 and 308 may be repeated a pre-determined number of iterations (e.g., 20) to yield higher accuracy.

If the terminating condition is not met, at 312, processing module 106 updates the current position with the estimated position and repeats steps 304, 306 and 308 using the updated current position. If the terminating condition is met, at 314, processing module outputs the estimated position.

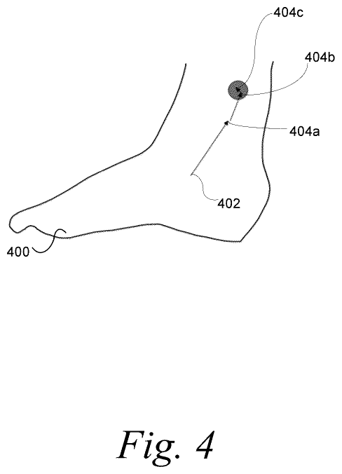

FIG. 4 illustrates the exemplary iterative process of estimating the position of a landmark on a patient's foot 400. The processing module 106 updates the current position from the initial position 402 to the next estimated positions (404a, 404b, 404c) of the landmark as the steps 304, 306 and 308 are repeated to find the target landmark. Estimations of the landmark position may be performed for a predetermined number of iterations or until a predetermined termination criteria is met.

Returning to FIG. 2, at 206, processing module 106 activates a feedback device 103 in response to the image quality measure. Due to the high computational efficiency of the present method, the feedback device 103 advantageously able to provide substantially real-time feedback to the user. The present position estimation has reduced the landmark estimation time to low milliseconds, thereby enabling immediate feedback and continuously fast scout image acquisition. The feedback device 103 may provide feedback in response to the image quality measure. As discussed previously, the image quality measure may be determined based on the patient's estimated position. The feedback device 103 may be positioned in or near the medical imaging device 102 to provide feedback in response to whether the patient is in the desired position or not.

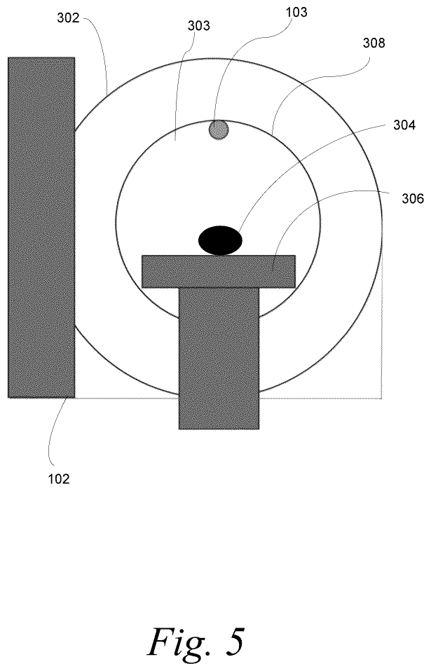

FIG. 5 shows a cross-sectional view of an exemplary medical imaging device 102. Medical imaging device 102 includes a magnetic resonance imaging (MRI) scanner gantry 302. The gantry 302 may include, for example, an X-ray source that projects a beam of x-rays toward a detector array on the opposite side of gantry 302. During a scan to acquire medical image data, gantry 302 and the components mounted thereon may rotate about a center of rotation. The gantry 302 includes an inner wall 308 that defines a chamber 303. Patient 304 is positioned on a moveable patient support apparatus 306 in the chamber 303. An operator may supply commands and parameters to a computer to provide control signals and information to control the movement of patient support table 306 and the patient positioned thereon. Particularly, patient support apparatus 306 moves portions of patient 304 through gantry chamber 303.

A feedback device 103 may be provided on the inner wall 308 of the gantry 302 to provide feedback to the operator in response to whether the patient is in the desired position or not. In some implementations, the feedback device 103 is a light emitting diode (LED) lamp positioned directly above the patient support table 306 in the gantry 302. The feedback device 103 may emit a combination of different light colors (e.g., red and green) in response to the image quality measure. For example, if the patient's position is estimated to be at or near the desired position, the light may turn from red to green, indicating that the patient is in the correct position and/or has stopped moving. Alternatively, or in combination thereof, feedback device 103 may be a sound device that generates an audible alert (e.g., beep, alarm) until the patient is in a desired position and/or has stopped moving. Other types of feedback devices, locations of the feedback device or image quality measures are also useful.

Returning to FIG. 2, at 208, processing module 106 determines whether the image quality measure satisfies a predetermined criterion. For example, the image quality measure may indicate whether the patient's position is at the desired position. If the image quality measure does not satisfy a predetermined criterion, the method 200 repeats step 202 to further receive additional volumetric images of the patient.

If the image quality measure satisfies a predetermined criterion, at 210, processing module 106 initiates a scan of the patient. For example, once the feedback device 103 shows a green light, an operator may start the scout scan of the patient and identify scanning ranges for a full scan. At the end, a full scan may be performed to obtain the desired best quality medical images.

The following is a list of non-limiting illustrative embodiments disclosed herein:
Illustrative embodiment 1. A system for providing imaging feedback, comprising: a feedback device positioned at a medical imaging device; a non-transitory memory device for storing computer readable program code; and a processor in communication with the non-transitory memory device and the feedback device, the processor being operative with the computer readable program code to perform steps including receiving a volumetric image of a patient from the medical imaging device, determining an image quality measure of the volumetric image, and activating the feedback device in response to the image quality measure.
Illustrative embodiment 2. The system of illustrative embodiment 1 wherein the feedback device comprises a visible feedback device, an audible feedback device, a tactile feedback device, or a combination thereof.
Illustrative embodiment 3. The system of any one of illustrative embodiments 1-2 wherein the feedback device is positioned in a gantry of the medical imaging device.
Illustrative embodiment 4. The system of any one of illustrative embodiments 1-3 wherein the image quality measure is based on a distance of an estimated position of the patient from a desired position.
Illustrative embodiment 5. The system of illustrative embodiment 4 wherein the feedback device comprises a light emitting diode lamp that turns from a first color to a second color in response to the patient being in the desired position.
Illustrative embodiment 6. The system of illustrative embodiment 4 wherein the feedback device comprises a sound device that beeps until the patient is in the desired position.
Illustrative embodiment 7. The system of any one of illustrative embodiments 1-6 wherein the image quality measure is based on presence of motion artifacts in the volumetric image.
Illustrative embodiment 8. An imaging feedback method, comprising: receiving a volumetric image of a patient from a medical imaging device; determining an image quality measure of the volumetric image; and activating a feedback device in response to the image quality measure.
Illustrative embodiment 9. The imaging feedback method of illustrative embodiment 8 wherein determining the image quality measure comprises determining presence of motion artifacts in the volumetric image.
Illustrative embodiment 10. The imaging feedback method of any one of illustrative embodiments 8-9 wherein determining the image quality measure comprises: determining, based on the volumetric image, an estimated position of the patient; and determining a distance of the estimated position of the patient from a desired position.
Illustrative embodiment 11. The imaging feedback method of illustrative embodiment 10 wherein determining, based on the volumetric image, the estimated position of the patient comprises: (i) providing a sparse sampling model for sparse sampling around a current position in the volumetric image; (ii) sampling voxels from the volumetric image around the current position using the sparse sampling model to obtain sparse sampling descriptors; and (iii) determining the estimated position of the patient by applying a trained regression model to the sparse sampling descriptors.
Illustrative embodiment 12. The imaging feedback method of illustrative embodiment 11 wherein the current position is initially set to a center of the volumetric image.
Illustrative embodiment 13. The imaging feedback method of illustrative embodiment 11 wherein providing the sparse sampling model for sparse sampling comprises providing the sparse sampling model such that voxels are sampled with a sampling rate which decreases with a distance of a respective voxel from the current position.
Illustrative embodiment 14. The imaging feedback method of illustrative embodiment 11 wherein the trained regression model comprises a trained residual neural network.
Illustrative embodiment 15. The imaging feedback method of illustrative embodiment 11 wherein the trained regression model generates normalized coordinates of at least one anatomical landmark indicative of the estimated position of the patient.
Illustrative embodiment 16. The imaging feedback method of any one of illustrative embodiments 8-15 further comprising repeating steps (i), (ii) and (iii) until a terminating condition is met.
Illustrative embodiment 17. The imaging feedback method of any one of illustrative embodiments 8-16 wherein activating the feedback device in response to the image quality measure comprises causing the feedback device to emit different light colors in response to the image quality measure.
Illustrative embodiment 18. The imaging feedback method of any one of illustrative embodiments 8-17 wherein activating the feedback device in response to the image quality measure comprises causing the feedback device to start or stop generating an audible alert in response to the image quality measure.
Illustrative embodiment 19. The imaging feedback method of any one of illustrative embodiments 8-18 further comprises initiating a scan of the patient in response to the image quality measure satisfying a predetermined criterion.
Illustrative embodiment 20. One or more non-transitory computer-readable media embodying instructions executable by machine to perform operations for providing imaging feedback, comprising: receiving a volumetric image of a patient from a medical imaging device; determining an image quality measure of the volumetric image; and activating a feedback device in response to the image quality measure.

The foregoing examples have been provided merely for the purpose of explanation and are in no way to be construed as limiting of the present framework disclosed herein. While the invention has been described with reference to various embodiments, it is understood that the words, which have been used herein, are words of description and illustration, rather than words of limitation. Further, although the invention has been described herein with reference to particular means, materials, and embodiments, the invention is not intended to be limited to the particulars disclosed herein, rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention in its aspects.

What is claimed is:

1. A system for providing imaging feedback, comprising:
a feedback device positioned at a medical imaging device;
a non-transitory memory device for storing computer readable program code; and
a processor in communication with the non-transitory memory device and the feedback device, the processor being operative with the computer readable program code to perform steps including
receiving a volumetric image of a patient from the medical imaging device,
determining an image quality measure of the volumetric image, and
activating the feedback device in response to the image quality measure.

2. The system of claim 1 wherein the feedback device comprises a visible feedback device, an audible feedback device, a tactile feedback device, or a combination thereof.

3. The system of claim 1 wherein the feedback device is positioned in a gantry of the medical imaging device.

4. The system of claim 1 wherein the image quality measure is based on a distance of an estimated position of the patient from a desired position.

5. The system of claim 4 wherein the feedback device comprises a light emitting diode lamp that turns from a first color to a second color in response to the patient being in the desired position.

6. The system of claim 4 wherein the feedback device comprises a sound device that beeps until the patient is in the desired position.

7. The system of claim 1 wherein the image quality measure is based on presence of motion artifacts in the volumetric image.

8. An imaging feedback method, comprising:
receiving a volumetric image of a patient from a medical imaging device;
determining an image quality measure of the volumetric image; and
activating a feedback device in response to the image quality measure.

9. The imaging feedback method of claim 8 wherein determining the image quality measure comprises determining presence of motion artifacts in the volumetric image.

10. The imaging feedback method of claim 8 wherein determining the image quality measure comprises:
determining, based on the volumetric image, an estimated position of the patient; and
determining a distance of the estimated position of the patient from a desired position.

11. The imaging feedback method of claim 10 wherein determining, based on the volumetric image, the estimated position of the patient comprises:
(i) providing a sparse sampling model for sparse sampling around a current position in the volumetric image;
(ii) sampling voxels from the volumetric image around the current position using the sparse sampling model to obtain sparse sampling descriptors; and
(iii) determining the estimated position of the patient by applying a trained regression model to the sparse sampling descriptors.

12. The imaging feedback method of claim 11 wherein the current position is initially set to a center of the volumetric image.

13. The imaging feedback method of claim 11 wherein providing the sparse sampling model for sparse sampling comprises providing the sparse sampling model such that voxels are sampled with a sampling rate which decreases with a distance of a respective voxel from the current position.

14. The imaging feedback method of claim 11 wherein the trained regression model comprises a trained residual neural network.

15. The imaging feedback method of claim 11 wherein the trained regression model generates normalized coordinates of at least one anatomical landmark indicative of the estimated position of the patient.

16. The imaging feedback method of claim 8 further comprising repeating steps (i), (ii) and (iii) until a terminating condition is met.

17. The imaging feedback method of claim 8 wherein activating the feedback device in response to the image quality measure comprises causing the feedback device to emit different light colors in response to the image quality measure.

18. The imaging feedback method of claim 8 wherein activating the feedback device in response to the image quality measure comprises causing the feedback device to start or stop generating an audible alert in response to the image quality measure.

19. The imaging feedback method of claim 8 further comprises initiating a scan of the patient in response to the image quality measure satisfying a predetermined criterion.

20. One or more non-transitory computer-readable media embodying instructions executable by machine to perform operations for providing imaging feedback, comprising:
receiving a volumetric image of a patient from a medical imaging device;
determining an image quality measure of the volumetric image; and
activating a feedback device in response to the image quality measure.
